(19) United States
(12) Patent Application Publication
(10) Pub. No.: US 2022/0168350 A1
(43) Pub. Date: Jun. 2, 2022

(54) Lung Cancer Specific Marrow Infiltrating Lymphocytes and Uses Thereof

(71) Applicants: WINDMIL THERAPEUTICS, INC., Baltimore, MD (US); THE JOHNS HOPKINS UNIVERSITY, Baltimore, MD (US)

(72) Inventors: Kimberly A. Noonan, Baltimore, MD (US); Ivan Borrello, Baltimore, MD (US)

(73) Assignees: WINDMIL THERAPEUTICS, INC., Baltimore, MD (US); THE JOHNS HOPKINS UNIVERSITY, Baltimore, MD (US)

(21) Appl. No.: 17/441,535

(22) PCT Filed: Mar. 20, 2020
(86) PCT No.: PCT/US20/23926

§ 371 Date: Sep. 21, 2021

Related U.S. Application Data
(60) Provisional application No. 62/822,207, filed on Mar. 22, 2019.

(51) Int. Cl. A61K 35/17; C12N 5/0783

(52) U.S. Cl. CPC A61K 35/17; C12N 2500/02; C12N 2501/2302; C12N 5/0636

(57) ABSTRACT

Lung cancer specific marrow infiltrating lymphocytes (“MILs”) and methods for making and using the same are described. Disclosed herein is a method for treating a subject having lung cancer with marrow infiltrating lymphocytes, the method comprising the steps of: (a) culturing a bone marrow sample obtained from the subject having lung cancer with an anti-CD3 antibody and an anti-CD28 antibody in a hypoxic environment to produce hypoxic-activated marrow infiltrating lymphocytes; (b) culturing the hypoxic-activated marrow infiltrating lymphocytes in a normoxic environment to produce the therapeutic activated marrow infiltrating lymphocytes; and (c) administering the therapeutic activated marrow infiltrating lymphocytes to the subject having lung cancer.

### GENERAL FIELD

The disclosure generally refers to marrow infiltrating lymphocytes (MILs) specific for treating lung cancer and methods of use thereof.

### BACKGROUND

Lung cancer is one of the most commonly diagnosed cancers and is a leading cause of cancer-related deaths, and new therapies remain a clinical priority. Recently, atezolizumab, an immune checkpoint inhibitor, was approved for treatment of nonsquamous non-small cell lung cancer, also referred to as NSCLC, for populations of patients with specific genetic fingerprints and to be administered in combination with standard chemotherapy regimens. Another approved immunotherapy for NSCLC is pembrolizumab, but again, is only effective for patients with specific genetic criteria. These therapies are only approved to be administered in combination with traditional chemotherapy with all of its inherent side effects.

In contrast, autologous cellular immunotherapy is an exciting area of research in cancer therapy, including metastatic lung cancer. The ability to eradicate measurable disease with adoptive T cell therapy requires T cells to be appropriately activated and present in sufficient numbers, possess appreciable anti-tumor activity, home to the tumor site, effectively kill the tumor upon encounter, and persist over time.

The treatment paradigm for patients with NSCLC is changing rapidly with the development of regulatory approval of immunotherapies, in particular check point inhibitors (CPIs) of the programmed cell death-1 (PD-1/programmed cell death ligand (PD-L1) pathway (together termed PD-1 CPIs). This pathway includes two proteins called PD-1, which is expressed on the surface of immune cells, and PD-L1, which is expressed on cancer cells. PD-L1 is upregulated on many cancer and other cells in the tumor microenvironment, allowing cancer cells to escape T cell-mediated tumor-specific immunity. When PD-1 and PD-L1 join together, the immune response is suppressed and the cancer cells can avoid detection. Treatment with PD-1 CPIs target this pathway by blocking the cancer cell's ability to suppress the immune response thereby allowing for “re-activation” of tolerized T cells, thus allowing the immune response to function properly. NSCLC is characterized by both high tumor neoantigen expression and high tumor mutation burden (Nowicki T. S. et al., Cancer J. 2018(January/February); 24(1):47-53)), each of which is associated with improved response to PD-1 CPIs. Therefore, treatment options for patients with NSCLC who progress after platinum-based chemotherapy (second line and beyond) include docetaxel (with or without ramucirumab), pemetrexed; and one of the four PD-1 CPIs currently approved for the treatment of metastatic NSCLC (nivolumab, pembrolizumab, durvalumab and atezolizumab), with pembrolizumab approved for first line therapy.

Marrow infiltrating lymphocytes (MILs™) are the product of activating and expanding bone marrow T cells (see Noonan et al., Cancer Res. (2005) 65(5): 2026-2034). The bone marrow is a specialized niche in the immune system which is enriched for antigen experienced, central memory T cells. MILs have been shown to confer immunologically measurable clinical benefits in patients with multiple myeloma (See U.S. Pat. No. 9,687,510). The bone marrow microenvironment has also been shown to harbor tumor-antigen specific T cells in patients with solid tumors such as breast, pancreatic and ovarian cancers (Schmitz-Winnenthal F. H. et al., Cancer Res. 2005 (November 1); 65(21):10079-1008). Therefore, development of lung cancer specific MILs for use in cancer therapy, possibly in conjunction with anti-PD1 CPIs and/or traditional chemotherapy, represents an exciting development.

### SUMMARY

Disclosed herein is a method for treating a subject having lung cancer with marrow infiltrating lymphocytes, the method comprising the steps of: (a) culturing a bone marrow sample obtained from the subject having lung cancer with an anti-CD3 antibody and an anti-CD28 antibody in a hypoxic environment to produce hypoxic-activated marrow infiltrating lymphocytes; (b) culturing the hypoxic-activated marrow infiltrating lymphocytes in a normoxic environment to produce the therapeutic activated marrow infiltrating lymphocytes; and (c) administering the therapeutic activated marrow infiltrating lymphocytes to the subject having lung cancer.

Also disclosed herein is an embodiment of the method as described above, wherein the hypoxic environment has an oxygen content of about 0% to about 5% oxygen.

Also disclosed herein is an embodiment of the method as described above, wherein the lymphocytes are cultured in the presence of IL-2.

Also disclosed herein is an embodiment of to provide the method as described above, wherein the culturing the hypoxic-activated marrow infiltrating lymphocytes in a normoxic environment is performed in the presence of IL-2.

Also disclosed herein is an embodiment of the method as described above, wherein the bone marrow sample is cultured in the hypoxic environment for about 24 hours.

Also disclosed herein is an embodiment of the method as described above, wherein the bone marrow sample is cultured in the hypoxic environment for about 2 days.

Also disclosed herein is an embodiment of the method as described above, wherein the bone marrow sample is cultured in the hypoxic environment for about 3 days.

Also disclosed herein is an embodiment of the method as described above, wherein the bone marrow sample is cultured in the hypoxic environment for about 2 to about 5 days.

Also disclosed herein is an embodiment of the method as described above, wherein the hypoxic environment is about 1% to about 2% oxygen.

Also disclosed herein is an embodiment of the method as described above, wherein the hypoxic-activated marrow infiltrating lymphocytes are cultured in the normoxic environment for about 2 to about 14 days.

Also disclosed herein is an embodiment of the method as described above, wherein the hypoxic-activated marrow infiltrating lymphocytes are cultured in the normoxic environment for about 6 days.

Also disclosed herein is an embodiment of the method as described above, wherein the hypoxic-activated marrow infiltrating lymphocytes are cultured in the normoxic environment for about 9 days.

Also disclosed herein is an embodiment of the method as described above, further comprising the step of removing a bone marrow sample from a subject having cancer prior to step (a).

Also disclosed herein is an embodiment of the method as described above, wherein the anti-CD3 antibody and the anti-CD28 antibody are bound on a bead.

Also disclosed herein is an embodiment of the method as described above, wherein the lung cancer is non small-cell lung cancer.

Also disclosed herein is a method for treating a subject having lung cancer with therapeutic activated marrow infiltrating lymphocytes, the method comprising the steps of: (a) culturing a bone marrow sample obtained from the subject having lung cancer with anti-CD3/anti-CD28 beads in a hypoxic environment of about 1% to about 2% oxygen for about 2 to about 5 days to produce hypoxic-activated marrow infiltrating lymphocytes; (b) culturing the hypoxic-activated marrow infiltrating lymphocytes in a normoxic environment of about 21% oxygen for about 2 to about 12 days in the presence of IL-2 to produce the therapeutic activated marrow infiltrating lymphocytes; and (c) administering the therapeutic activated marrow infiltrating lymphocytes to the subject having lung cancer.

Also disclosed herein is a method of treating lung cancer in a subject, the method comprising administering a pharmaceutical composition comprising lung cancer specific marrow infiltrating lymphocyte to the subject.

Also disclosed herein is an embodiment of the method as described above, wherein the lung cancer specific marrow infiltrating lymphocyte is obtained from a subject having lung cancer.

Also disclosed herein is an embodiment of the method as described above, wherein the lung cancer specific marrow infiltrating lymphocyte is autologous to the subject being treated.

Also disclosed herein is an embodiment of the method as described above, wherein the lung cancer specific marrow infiltrating lymphocyte is allogeneic to the subject being treated.

Also disclosed herein is an embodiment of the method as described above, wherein the marrow infiltrating lymphocyte is hypoxic activated.

Also disclosed herein is an embodiment of the method as described above, wherein the marrow infiltrating lymphocyte is hypoxic activated and normoxic activated.

Also disclosed herein is an embodiment of the method as described above, wherein the pharmaceutical composition is administered by parenteral administration, intraperitoneal or intramuscular administration.

Also disclosed herein is an embodiment of the method as described above, wherein the pharmaceutical composition is administered directly into the lung of the subject.

Also disclosed herein is an embodiment of the method as described above, wherein about 75% to about 100% of the marrow infiltrating lymphocytes administered to the subject express CD3.

Also disclosed herein is an embodiment of the method as described above, wherein about 80% to about 100% of the marrow infiltrating lymphocytes administered to the subject express CD3.

Also disclosed herein is an embodiment of the method as described above, wherein about 85% to about 100% of the marrow infiltrating lymphocytes administered to the subject express CD3.

Also disclosed herein is an embodiment of the method as described above, wherein about 90% to about 100% of the marrow infiltrating lymphocytes administered to the subject express CD3.

Also disclosed herein is an embodiment of the method as described above, wherein the ratio of CD4⁺:CD8⁺ T cells present in the composition or MILs administered to the subject is about 2:1.

Also disclosed herein is a composition comprising a population of hypoxic-activated marrow infiltrating lymphocytes isolated from a patient with lung cancer, wherein about 75% to about 100% of the population of the hypoxic activated marrow infiltrating lymphocytes expresses CD3.

Also disclosed herein is an embodiment of the composition as described above, wherein about 80% to about 100% of the population of the hypoxic activated marrow infiltrating lymphocytes expresses CD3.

Also disclosed herein is an embodiment of the composition as described above, wherein about 85% to about 100% of the population of the hypoxic activated marrow infiltrating lymphocytes expresses CD3.

Also disclosed herein is an embodiment of the composition as described above, wherein about 90% to about 100% of the population of the hypoxic activated marrow infiltrating lymphocytes expresses CD3.

Also disclosed herein is an embodiment of the composition as described above, wherein the ratio of CD4⁺:CD8⁺ T cells present in the composition is about 2:1.

Also disclosed herein is an embodiment of the composition as described above, wherein the cell population is obtainable from a bone marrow sample obtained from a subjecting having lung cancer by: (a) culturing the bone marrow sample with an anti-CD3 antibody and an anti-CD28 antibody in a hypoxic environment of about 1% to about 3% oxygen to produce activated marrow infiltrating lymphocytes; and (b) culturing the activated marrow infiltrating lymphocytes in a normoxic environment in the presence of IL-2 to produce the composition.

Also disclosed herein is an embodiment of the composition as described above, wherein the marrow infiltrating lymphocytes are lung cancer specific.

Also disclosed herein is an embodiment of the method as described above, wherein the subject has been subjected to treatment with anti-PD1 prior to obtaining the bone marrow sample.

### BRIEF DESCRIPTION OF THE DRAWINGS

FIG. 1 shows the percentage of expanded and activated tumor-specific MILs from 4 patients. For the assay MILs™ are carboxyfluorescein succinimidyl ester (CFSE)-labelled and co-cultured with autologous antigen-presenting cells pulsed with tumor lysates from either lung cell line lysates (assay group) or myeloma cell line lysates (negative control). Tumor-specific MILs™ were denoted as CD3⁺/CFSE^{low}/IFNγ⁺ cells. Data shown have been gated on both CD3 and CFSE^{low} and graphed is the IFN-γ production for either media alone (negative control) myeloma cell line lysate (negative control), or lung cell line lysates.

FIGS. 2 and 3 shows lung MILs specificity data each from different patients. Similar to the above-described assay, the MILs are CFSE-labelled and co-cultured with autologous antigen-presenting cells pulsed as labelled. Tumor-specific MILs are gated on either CD3, CD4, or CD8 alone, or both CD3, CD4, or CD8 and CFSE^{low}, and graphed according to IFN-γ production for either media alone (negative control), myeloma cell line lysate (negative control), or lung cell lysates.

FIG. 4 shows polyfunctionality of lung MILs from two different patients. MILs are co-cultured with autologous antigen-presenting cells with media alone (media), multiple myeloma cell line lysates (MM) or lung cancer cell line lysates (A549, H2170 & H460). Tumor-specific MILs are gated on either CD3, CD4 or CD3, CD8 and graphed according to IFN-γ (G), TNFa (A) and GrzB (GrB) production for either media alone (negative control), myeloma cell line lysate (negative control), or lung cell lysates.

FIG. 5 shows lung MILs specificity data for the first patient treated on NSCLC trial. MILs are co-cultured with autologous antigen-presenting cells. Tumor-specific MILs are gated on either CD3, CD4 and graphed according to IFN-γ production for either media alone (negative control), myeloma cell line lysate (negative control), or lung cell lysates. Ratios compare the MILs product administered to the patient (CGN) to three research-grade products (WM).

FIG. 6 shows lung MILs specificity data for the first patient treated on NSCLC trial. MILs are co-cultured with autologous antigen-presenting cells. Tumor-specific MILs are gated on either CD3, CD8 and graphed according to IFN-γ production for either media alone (negative control), myeloma cell line lysate (negative control), or lung cell lysates. Ratios compare the MILs product administered to the patient (CGN) with three different research-grade products (WM).

FIG. 7 shows polyfunctionality of lung MILs for the first NSCLC patient treated with MILs. MILs are co-cultured with autologous antigen-presenting cells. Tumor-specific MILs are gated on CD3, CD8 and graphed according to IFN-γ, GrzB, TNFa production for either media alone (negative control), myeloma cell line lysate (negative control), or lung cell lysates. Ratios compare the MILs product administered to the patient (CGN) with three different research-grade products (WM).

FIG. 8 shows polyfunctionality of lung MILs for the first NSCLC patient treated with MILs. MILs are co-cultured with autologous antigen-presenting cells. Tumor-specific MILs are gated on CD3, CD4 and graphed according to IFN-γ, GrzB, TNFa production for either media alone (negative control), myeloma cell line lysate (negative control), or lung cell lysates. Ratios compare the MILs product administered to the patient (CGN) with three different research-grade products (WM).

### DETAILED DESCRIPTION OF EXEMPLARY EMBODIMENTS

As used herein and unless otherwise indicated, the term “about” is intended to mean±5% of the value it modifies. Thus, “about 100” means 95 to 105. Additionally, the term “about” modifies a term in a series of terms, such as “about 1, 2, 3, 4, or 5” it should be understood that the term “about” modifies each of the members of the list, such that “about 1, 2, 3, 4, or 5” can be understood to mean “about 1, about 2, about 3, about 4, or about 5.” The same is true for a list that is modified by the term “at least” or other quantifying modifier, such as, but not limited to, “less than,” “greater than,” and the like.

As used herein and in the appended claims, the singular forms “a”, “an” and “the” include plural reference unless the context clearly dictates otherwise.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the terms “treat,” “treated,” or “treating” mean both therapeutic treatments wherein the object is to slow down (lessen) an undesired physiological condition, disorder or disease, or obtain beneficial or desired clinical results. For purposes of the embodiments described herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Thus, “treatment of cancer” or “treating cancer” means an activity that alleviates or ameliorates any of the primary phenomena or secondary symptoms associated with the cancer or any other condition described herein. In some embodiments, the cancer that is being treated is one of the cancers recited herein.

As used herein, the term “subject” can be used interchangeably with the term “patient”. The subject can be a mammal, such as a dog, cat, monkey, horse, or cow, for example. In some embodiments, the subject is a human. In some embodiments, the subject has been diagnosed with lung cancer. In some embodiments, the subject is believed to have lung cancer. In some embodiments, the subject is suspected of having lung cancer.

As used herein, the term “express” as it refers to a cell surface receptor, such as, but not limited to, CD3, CD4, and CD8, can also be referred to as the cell being positive for that marker. For example, a cell that expresses CD3 can also be referred to as CD3 positive (CD3+).

The term “cancer” as used herein is defined as disease characterized by the rapid and uncontrolled growth of aberrant cells. Cancer cells can spread locally or through the bloodstream and lymphatic system to other parts of the body. The term “lung cancer” as used herein is defined as cancer originating from the lung, or cancer on or within the lung. In some embodiments, lung cancer is small cell lung cancer, carcinoid tumor cancer, adenoid cystic carcinomas, hamartomas, lymphomas, sarcomas, or non-small cell lung cancer. In some embodiments, non-small cell lung cancer is squamous cell carcinoma, adenocarcinoma, or large cell carcinoma.

“Effective amount” or “therapeutically effective amount” are used interchangeably herein, and refer to an amount of a compound, formulation, material, or composition, as described herein effective to achieve a particular biological result. Such results may include, but are not limited to, the inhibition of virus infection as determined by any means suitable in the art.

As used herein, “marrow infiltrating lymphocytes” or “MILs” are a subpopulation of immune cells and are described for example in, U.S. Pat. No. 9,687,510, which is hereby incorporated by reference in its entirety. MILs significantly differ from peripheral blood lymphocytes (PBLs). For example, MILs are more easily expanded, upregulate activation markers to a greater extent than PBLs, maintain more of a skewed V3 repertoire, traffic to the bone marrow, and most importantly, possess significantly greater tumor specificity. In some embodiments, MILs can be activated, for example, by incubating them with anti-CD3/anti-CD-28 beads and under hypoxic conditions, as described herein. In some embodiments, growing MILs under hypoxic conditions is also described in U.S. Pat. No. 9,687,510, and International Application No. WO2016/037054, both of which are incorporated by reference herein in their entirety.

Compared to previous methods of using MILs in non-solid tumor type cancers such as multiple myeloma, use of MILs to treat solid tumor cancers require a different and distinct paradigm. Many tumors are known to exploit the PD-1/PD-L1 pathway through upregulation of PD-L1 on tumor cells and other cells to escape T cell-mediated tumor-specific immunity. Inhibiting the interaction between PD-1 and PD-L1 decreases this immunosuppressive signal, allowing tumor-specific cytotoxic T cells to access and kill the tumor cells. MILs™ are distinct from two other forms of adoptive cellular therapy. Table 5 compares key characteristics of MILs™ to chimeric antigen receptor (CAR)-T and genetically engineered T cell receptor (eTCR) cell therapies. Most critical is that the efficacy of CAR-T and eTCR cell therapies are dependent upon engagement of the cognate antigen on the tumor cells; selective editing or deletion of that antigen by the tumor will render CAR-T or eTCR therapies non-effective. In contrast, the polyclonal recognition of MILs™ should minimize the risk of generating antigen escape loss tumor variants as a mechanism of disease relapse.

TABLE 1
Comparison of MILs ™ to CAR-T and eTCR cells

| Characteristic | CAR-T/eTCR | MILs ™ |
|---|---|---|
| Cell Source | Peripheral Blood | Bone Marrow |
| Antigen Specificity | Monoclonal (Limited) | Polyclonal |
| Genetic Modification | Required | Not required |
| HLA Restricted | No (CAR-T); Yes (eTCR) | No |

Abbreviations:
MILs ™ = marrow infiltrating lymphocytes;
CAR-T = chimeric antigen receptor;
eTCR = engineered T cell receptor;
HLA = human leukocyte antigen

In some embodiments, methods to prepare MILs may include removing cells from the bone marrow, lymphocytes, and/or marrow infiltrating lymphocytes from the subject; incubating the cells in a hypoxic environment, thereby producing activated MILs. In some embodiments, the subject has cancer. The cells can also be activated in the presence of anti-CD3/anti-CD28 antibodies and cytokines as described herein.

Bone marrow may be collected from a patient having lung cancer that has been previously treated with a check point inhibitor. The checkpoint inhibitor can be an anti-PD-1 antibody, an anti-PD-L1 antibody, or a combination of these. The patient may have non-metastatic or metastatic disease at the time of the bone marrow removal. The patient may have squamous or non-squamous NSCLC. The patient may have been previously treated with chemotherapy or not.

The collected bone marrow may be frozen or immediately used, for example, to create tumor specific MILs. If the bone marrow is frozen, it is preferably thawed before incubation. The bone marrow may be treated to purify MILs through methods known to one of ordinary skill in the art. The MILs may be activated, for example, with beads, e.g., anti-CD4/CD28 beads. The ratio of beads to cells in the solution may vary; in some embodiments, the ratio is 3 to 1. Similarly, the MILs may be expanded in the presence of one or more antibodies, antigens, and/or cytokines, e.g., in the absence of anti-CD3/CD28 beads. The cell count for the collected bone marrow may be determined, for example, to adjust the amount of beads, antibodies, antigens, and/or cytokines to be added to the MILs. In some embodiments, MILs are captured using beads specifically designed to collect the cells.

The collected MILs can be grown in a hypoxic environment for a first period of time. The hypoxic environment may include less than about 7% oxygen, such as less than about 7%, 6%, 5%, 4%, 3%, 2%, or 1% oxygen. For example, the hypoxic environment may include about 0% oxygen to about 7% oxygen, 0% oxygen to about 6% oxygen, such as about 0% oxygen to about 5% oxygen, about 0% oxygen to about 4% oxygen, about 0% oxygen to about 3% oxygen, about 0% oxygen to about 2% oxygen, about 0% oxygen to about 1% oxygen. In some embodiments, the hypoxic environment includes about 1% to about 5% oxygen. In some embodiments, the hypoxic environment is about 1% to about 2% oxygen. In some embodiments, the hypoxic environment is about 0.5% to about 1.5% oxygen. In some embodiments, the hypoxic environment is about 0.5% to about 2% oxygen. The hypoxic environment may include about 7%, 6%, 5%, 4%, 3%, 2%, 1%, or about 0% oxygen, and all fractions thereof in between these amounts.

Incubating MILs in a hypoxic environment may include incubating the MILs, e.g., in tissue culture medium, for at least about 1 hour, such as at least about 12 hours, 18 hours, 24 hours, 30 hours, 36 hours, 42 hours, 48 hours, 60 hours, 3 days, 4 days, 5 days, 6 days, 7 days, 8 days, 9 days, 10 days, 11 days, 12 days, 13 days, or even at least about 14 days. Incubating may include incubating the MILs for about 1 hour to about 30 days, such as about 1 day to about 20 days, about 1 day to about 14 days, or about 1 day to about 12 days. In some embodiments, incubating MILs in a hypoxic environment includes incubating the MILs in a hypoxic environment for about 2 days to about 5 days. The method may include incubating MILs in a hypoxic environment for about 1 day, 2 days, 3 days, 4 days, 5 days, 6 days, 7 days, 8 day, 9 days, 10 days, 11 days, 12 days, 13 days, or 14 days. In some embodiments, the method includes incubating the MILs in a hypoxic environment for about 3 days. In some embodiments, the method includes incubating the MILs in a hypoxic environment for about 2 days to about 4 days. In some embodiments, the method includes incubating the MILs in a hypoxic environment for about 3 days to about 4 days.

In some embodiments, hypoxic-activated MILs are then cultured in a normoxic environment to produce the therapeutically activated marrow infiltrating lymphocytes. In some embodiments, the normoxic environment may include at least about 7% oxygen. In some embodiments, the normoxic environment may include about, such as about 8% oxygen to about 30% oxygen, 10% oxygen to about 30% oxygen, about 15% oxygen to about 25% oxygen, about 18% oxygen to about 24% oxygen, about 19% oxygen to about 23% oxygen, or about 20% oxygen to about 22% oxygen. In some embodiments, the normoxic environment includes about 21% oxygen.

In some embodiments, the MILs are cultured in the presence of IL-2 or other cytokines. In some embodiments, the MILs are cultured in normoxic conditions in the presence of IL-2. In some embodiments, the other cytokines can be IL-7, IL-15, IL-9, IL-21, or any combination thereof. In some embodiments, the MILs can be cultured in cell culture medium that includes one or more cytokines, e.g., such as IL-2, IL-7, and/or IL-15, or any suitable combination thereof. Illustrative examples of suitable concentrations of each cytokine or the total concentration of cytokines includes, but is not limited to, about 25 IU/mL, about 50 IU/mL, about 75 IU/mL, about 100 IU/mL, about 125 IU/mL, about 150 IU/mL, about 175 IU/mL, about 200 IU/mL, about 250 IU/mL, about 300 IU/mL, about 350 IU/mL, about 400 IU/mL, about 450 IU/mL, or about 500 IU/mL or any intervening amount. In some embodiments, the cells are cultured in about 100 IU/mL of each of, or in total of, IL-2, IL-1, and/or IL-15, or any combination thereof. In some embodiments, the cell culture medium includes about 250 IU/mL of each of, or in total of, IL-2, IL-1, and/or IL-15, or any combination thereof.

Incubating MILs in a normoxic environment may include incubating the MILs, for at least about 1 hour, such as at least about 12 hours, 18 hours, 24 hours, 30 hours, 36 hours, 42 hours, 48 hours, 60 hours, 3 days, 4 days, 5 days, 6 days, 7 days, 8 days, 9 days, 10 days, 11 days, 12 days, 13 days, or even at least about 14 days. Incubating may include incubating the MILs for about 1 hour to about 30 days, such as about 1 day to about 20 days, about 1 day to about 14 days, about 1 day to about 12 days, or about 2 days to about 12 days.

In some embodiments, the MILs are obtained by extracting a bone marrow sample from a subject and culturing/incubating the cells as described herein. In some embodiments, the bone marrow sample is centrifuged to remove red blood cells. In some embodiments, the bone marrow sample is not subject to apheresis. In some embodiments, the bone marrow sample does not include PBLs or the bone marrow sample is substantially free of PBLs. These methods select for cells that are not the same as what have become to be known as tumor infiltrating lymphocytes (“TILs”), which have distinct limitations for use in adoptive T cell therapy. Thus, a MIL is not a TIL. In some embodiments, the bone marrow sample contains less than 10%, 9%, 8%, 7%, 6%, 5%, 4%, 3%, 2%, or less than 1% PBLs as compared to the total of MILs. In some embodiments, the sample is free of PBLs.

In some embodiments, the cells are also activated by culturing with antibodies to CD3 and CD28. This can be performed, for example by incubating the cells with anti-CD3/anti-CD28 beads that are commercially available or that can be made by one of skill in the art. The cells can then be plated in a plate, flask, or bag. Hypoxic conditions can be achieved by flushing either the hypoxic chamber or cell culture bag for 3 minutes with a 95% Nitrogen and 5% CO₂ gas mixture. This can lead to, for example, 1-2% or less O₂ gas in the receptacle. Examples of such beads and methods of stimulation can be found, for example, in U.S. Pat. Nos. 6,352,694, 6,534,055, 6,692,964, 6,797,514, 6,867,041, and 6,905,874, each of which is incorporated by reference in its entirety. Alternatives to beads include engineered cells, such as K562 cells, that can be used to stimulate the MILs. Such methods can be found in, for example, U.S. Pat. Nos. 8,637,307 and 7,638,325, each of which is incorporated by reference in its entirety. Cells can also be stimulated using other methods, such as those described in U.S. Pat. No. 8,383,099, which is incorporated by reference in its entirety.

In some embodiments, activated MILs and/or therapeutic activated MILs are administered to a subject having, or suspected of having, lung cancer. In some embodiments, hypoxic-activated MILs and/or therapeutic activated MILs are produced from a bone marrow sample from a subject having or suspected of having lung cancer, then administering to the same subject to treat lung cancer. In some embodiments, the MILs are allogeneic to the subject.

In some embodiments, the MILs can be administered in a pharmaceutical preparation or pharmaceutical composition. Pharmaceutical compositions including the lung cancer specific MILs may further include buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions can be formulated for parenteral administration, e.g., intravascular (intravenous or intraarterial), intraperitoneal or intramuscular administration. In some embodiments, the MILs and/or compositions are administered by parenteral administration, e.g., intravascular (intravenous or intraarterial), intraperitoneal or intramuscular administration. The compositions can also be administered directly into the lung or into the tumor. In some embodiments, the compositions are administered intravenously.

In some embodiments, compositions, whether they be solutions, suspensions or other like form, may include one or more of the following: DMSO, sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

In some embodiments, the subject can be pre-conditioned with cyclophosphamide with or without fludarabine. One such example is provided for in U.S. Pat. No. 9,855,298, which is hereby incorporated by reference. Another non-limiting example is administering fludarabine (30 mg/m² intravenous daily for 3 days) and cyclophosphamide (300 mg/m² intravenous daily for 3 days starting with the first dose of fludarabine). After administration, the MILs™ can be administered after, e.g., 2 to 14 days after, completion of the fludarabine and cyclophosphamide. In some embodiments, the cyclophosphamide is administered or 2-3 days at a dose of about 300 to about 600 mg/m².

In some embodiments, the pharmaceutical composition that is administered includes lung cancer-specific MILs as provided for herein. A composition of such MILs is also provided for herein. In some embodiments, the lung cancer specific MILs are hypoxic activated. In some embodiments, the lung cancer-specific MILs are hypoxic activated/normoxic activated MILs. A lung cancer-specific MIL is a MIL that can specifically target lung cancer in a subject.

In some embodiments, the composition includes a population of lung cancer specific MILs that are CD3 positive. In some embodiments, at least about, or at least, 40% of the MILs are CD3 positive. In some embodiments, about, or at least, 45%, 50%, 55%, 60%, 65%, 70%, 75%, 80%, 85%, 86%, 87%, 88%, or 89% of MILs are CD3 positive. In some embodiments, at least, or about, 80% of the MILs are CD3 positive. In some embodiments, about 40% to about 100% of the MILs are CD3 positive. In some embodiments, about 45% to about 100%, about 50% to about 100%, about 55% to about 100%, about 60% to about 100%, about 65% to about 100%, about 70% to about 100%, about 75% to about 100%, about 80% to about 100%, about 85% to about 100%, about 86% to about 100%, about 87% to about 100%, about 88% to about 100%, or about 90% to about 100% of the MILs are CD3 positive (express CD3).

In some embodiments, the composition includes either a population of MILs that do not express CD3, or a population of MILs that expresses low levels of CD3, for example, relative to the expression level of MILs from the population of MILs that express CD3.

In some embodiments, the composition includes a population of MILs that expresses interferon gamma (“IFNγ”), i.e., wherein each cell in the population of MILs that expresses IFNγ is a marrow infiltrating lymphocyte that expresses IFNγ, e.g., as detected by flow cytometry. For example, at least about 2% of the cells in the composition may be MILs that express IFNγ, or at least about 2%, 3%, 4%, 5%, 6%, 7%, 8%, 9%, 10%1, 1%1, 2%1, 3%1, 14%, 15%, 16%, 17%, or even at least about 18% of the MILs express IFNγ. In some embodiments, about 2% to about 100% of the MILs express IFNγ, such as about 2% to about 100%, about 3% to about 100%, about 4% to about 100%, about 5% to about 100%, about 6% to about 100%, about 7% to about 100%, about 8% to about 100%, about 9% to about 100%, about 10% to about 100%, about 11% to about 100%, about 12% to about 100%, about 13% to about 100%, about 14% to about 100%, about 15% to about 100%, about 16% to about 100%, about 17% to about 100%, or even about 18% to about 100% of the MILs. In some embodiments, the composition includes either a population of MILs that do not express IFNγ, e.g., as detected by flow cytometry, or a population of MILs that expresses low levels of IFNγ, i.e., relative to the expression level of MILs from the population of MILs that express IFNγ.

In some embodiments, the composition includes a population of MILs that expresses CXCR4. For example, at least about 98% of the MILs express CXCR4, such as at least about 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, or even at least about 99.7% of the MILs. In some embodiments, about 98% to about 100% may be MILs that express CXCR4, such as at least about 98.1% to about 100%, about 98.2% to about 100%, about 98.3% to about 100%, about 98.4% to about 100%, about 98.5% to about 100%, about 98.6% to about 100%, about 98.7% to about 100%, about 98.8% to about 100%, about 98.9% to about 100%, about 99.0% to about 100%, about 99.1% to about 100%, about 99.2% to about 100%, about 99.3% to about 100%, about 99.4% to about 100%, about 99.5% to about 100%, about 99.6% to about 100%, or even about 99.7% to about 100% of the MILs in the composition. In some embodiments, the composition includes either a population of MILs that do not express CXCR4, e.g., as detected by flow cytometry, or a population of MILs that expresses low levels of CXCR4, i.e., relative to the expression level of MILs from the population of MILs that express CXCR4.

In some embodiments, the composition includes a population of MILs that expresses CD4. The population of MILs that expresses CD4 may include a plurality of MILs that expresses CXCR4.

The population of MILs that expresses CD4 may include a plurality of MILs that expresses 4-1BB. For example, at least about 21% of the cells in the composition may be MILs from the plurality of MILs that expresses 4-1BB, such as at least about 22%, 23%, 24%, 25%, 26%, 27%, 28%, 29%, 30%, 31%, 32%, 33%, 34%, 35%, 36%, 37%, 38%, 39%, 40%, 41%, 42%, or even at least about 43% of the cells in the composition. In some embodiments, about 21% to about 100% of the cells in the composition may be MILs from the plurality of MILs that expresses 4-1BB, such as about 22% to about 100%, about 23% to about 100%, about 24% to about 100%, about 25% to about 100%, about 26% to about 100%, about 27% to about 100%, about 28% to about 100%, about 29% to about 100%, about 30% to about 100%, about 31% to about 100%, about 32% to about 100%, about 33% to about 100%, about 34% to about 100%, about 35% to about 100%, about 36% to about 100%, about 37% to about 100%, about 38% to about 100%, about 39% to about 100%, about 40% to about 100%, about 41% to about 100%, about 42% to about 100%, or even about 43% to about 100% of the cells in the composition.

The composition may include a population of MILs that expresses CD8. The population of MILs that expresses CD8 may include a plurality of MILs that expresses CXCR4.

The population of MILs that expresses CD8 may include a plurality of MILs that expresses 4-1BB. For example, at least about 21% of the cells in the composition may be MILs from the plurality of MILs that expresses 4-1BB, such as at least about 8%, 9%, 10%, 11%, 12%, 13%, 14%, 15%, 16%, 17%, 18%, 19%, 20% or even at least about 21% of the cells in the composition. In some embodiments, about 2% to about 100% of the cells in the composition may be MILs from the plurality of MILs that expresses 4-1BB, such as about 8% to about 100%, about 9% to about 100%, about 10% to about 100%, about 11% to about 100%, about 12% to about 100%, about 13% to about 100%, about 14% to about 100%, about 15% to about 100%, about 16% to about 100%, about 17% to about 100%, about 18% to about 100%, about 19% to about 100%, about 20% to about 100%, or even about 21% to about 100% of the cells in the composition.

In some embodiments, the composition includes a population of MILs that expresses 4-1BB. For example, at least about 21% of the cells in the composition may be MILs from the population of MILs that expresses 4-1BB, such as at least about 22%, 23%, 24%, 25%, 26%, 27%, 28%, 29%, 30%, 31%, 32%, 33%, 34%, 35%, 36%, 37%, 38%, 39%, 40%, 41%, 42%, or even at least about 43% of the cells in the composition. In some embodiments, about 21% to 100% of the cells in the composition may be MILs from the population of MILs that expresses 4-1BB, such as about 22% to about 100%, about 23% to about 100%, about 24% to about 100%, about 25% to about 100%, about 26% to about 100%, about 27% to about 100%, about 28% to about 100%, about 29% to about 100%, about 30% to about 100%, about 31% to about 100%, about 32% to about 100%, about 33% to about 100%, about 34% to about 100%, about 35% to about 100%, about 36% to about 100%, about 37% to about 100%, about 38% to about 100%, about 39% to about 100%, about 40% to about 100%, about 41% to about 100%, about 42% to about 100%, or even about 43% to about 100% of the cells in the composition. In some embodiments, the composition includes either a population of MILs that do not express 4-1BB, e.g., as detected by flow cytometry, or a population of MILs that expresses low levels of 4-1BB, i.e., relative to the expression level of MILs from the population of MILs that express 4-1BB.

In some embodiments, the composition includes MILs that express CD4. In some embodiments, the composition includes MILs that express CD8. In some embodiments, the ratio of CD4⁺:CD8⁺ MILs present in the composition is about 2:1.

The composition may include a population of MILs that expresses CD8. The population of MILs that expresses CD8 may include a plurality of MILs that expresses CXCR4.

In some embodiments, the composition includes a population of MILs that expresses CD4. The population of MILs that expresses CD4 may include a plurality of MILs that expresses CXCR4.

The MILs may express the different factors or surface receptors as described herein alone or in combination with one another. Thus, for example, a MIL can be CD3+, CD4+, and/or CD8+. Such cells can also express IFNγ. The cells can also be positive or negative for the various factors or receptors provided for herein.

In some embodiments, the methods for preventing or treating lung cancer in a subject are provided. In some embodiments, the methods include administering to a subject one of the compositions described herein, such as, but not limited to, lung-specific MILs as provided herein. In some embodiments, the compositions are administered as provided for herein. In some embodiments, the method includes administering to the subject a therapeutically effective amount of any one of the compositions described herein. In some embodiments, the method includes administering to the subject a therapeutically-effective amount of the lung cancer specific MILs. In some embodiments, the MILs are activated. In some embodiments, the MILs are hypoxic-activated as described herein and referenced herein. In some embodiments, the MILs are cultured under hypoxic conditions followed by normoxic conditions as described and referenced herein. In some embodiments, MILs are obtained or extracted from a bone marrow sample obtained from a subject having lung cancer. In some embodiments, the MILs are allogeneic to the subject being treated. In some embodiments, the methods include (a) culturing a bone marrow sample from a subject with an anti-CD3 antibody and an anti-CD28 antibody in a hypoxic environment of about 1% to about 3% oxygen to produce activated marrow infiltrating lymphocytes; and (b) culturing the activated marrow infiltrating lymphocytes in a normoxic environment in the presence of IL-2 to produce the composition. The composition can be then be administered to the subject with lung cancer.

The following examples are illustrative, but not limiting, of the compositions and methods described herein. Other suitable modifications and adaptations known to those skilled in the art are within the scope of the following embodiments.

### EXAMPLES

### Example 1: Producing Tumor Specific MILs from Subjects with Lung Cancer

### Bone Marrow Mononuclear Cell Separation

Bone marrow samples were collected from the iliac crest under IRB approved informed consent from Non-Small Cell Lung Cancer (“NSCLC”) patients (n=4) previously treated with anti-PD1. Red blood cells were removed from the bone marrow using a ficoll gradient. Bone marrow mononuclear cells were frozen at a 10×10⁶ cells/ml in liquid nitrogen until the time of activation and expansion.

### Activation and Expansion of Bone Marrow T Cells

Previously frozen bone marrow mononuclear cells were slow thawed; and enumerated for both number and viability using a hemocytometer and trypan blue. Approximately 1 million cells were obtained for flow cytometry to determine the percentage of CD3. Briefly thawed cells were washed 2× in FACS buffer (1×HBSS containing 2% FBS and sodium azide). Cells were extracellularly stained with Live/Dead Violet, Glycophorin A (GlyA), CD3, CD4 and CD8 for 10 minutes at room temperature. The cells were washed 2× with FACS buffer and resuspended in 100 ul of FACs buffer. The cells were run on a Beckman Coulter Navios flow cytometer and analyzed with Kaluza software. The percentage of CD3 were determined as live/GlyA negative/CD3 positive.

This percentage of CD3 was used to determine the number of anti-CD3/anti-CD28 beads to add to the culture. For each tumor type, the optimal bead:T cell ratio was determined. Beads were added to the thawed mononuclear bone marrow cells at the optimized ratio. IL2 was added at 100 U/ml and cells were plated in 96 well round bottom plates, 200 μl/well, 1×10⁶ total cells/ml for a total of 2×10⁵ cells/well.

Plates were placed in a hypoxic chamber. Oxygen was flushed out of the chamber for 2 minutes by having one valve of the chamber open and one valve connected to 95% nitrogen and 5% CO₂. After 2 min the open valve was closed and the chamber was filled with the gas mixture for 30 seconds. The second valve was closed and the chamber was placed in a 37° C./5% CO₂ incubator for 3 days. On the third day the chamber was open and media containing 100 U/ml of IL2 was added.

Cells were maintained for the remainder of the culture in 37° C./5% CO₂. Cells were evaluated at all consecutive days of culture to determine if the culture needs to be split. When the cells were split, IL2 was replenished for a total of 100 U/ml IL2. Cells were grown from 5-10 days as needed to have enough cells for experimentation. Cells in these experiments were grown for 7 days.

On the final day of culture a magnet was used to remove the anti-CD3/anti-CD28 beads. The cells were washed and enumerated for number and viability using a hemacytometer and Trypan Blue.

The percentage of CD3 was determined as described above. Fold expansion of CD3 was determined as (total cells x % CD3)/total number of CD3 cells at start of expansion. The fold expansion was verified in two separate experiments and cells were grown to both 7 and 10 days. In the first experiment with CD3 cells grown for 7 days, a range of 22-fold expansion to 99-fold expansion for the 4 patients was seen with a mean of approximately 60-fold. In the second 7-day experiment for the 4 patients, there was a low of 23-fold expansion with a high of 107-fold expansion with a mean of approximately 65-fold. Therefore, the results of the two 7-day experiments are very similar. When CD3 cells were grown for 10 days in two similar experiments with the same patients, the ranges of fold expansion were wider in both experiments, with means of approximately 90-fold and 125-fold. MILs were frozen at 10×10⁶ ml in 1N2 until needed for further experimentation.

### Flow Cytometry on MILs

Cells were slow thawed and enumerated for number and viability. Cells were washed 2× with FACS buffer (as before) and stained extra-cellularly with Live/Dead Violet, CD3, CD4, CD8, CD45RO and CD62L for 10 minutes at room temperature. The cells were washed 2× with FACS buffer and resuspended in 100 ul of FACs buffer. The cells were run on a Beckman Coulter Navios flow cytometer and analyzed with Kaluza software. Percentage of CD8 was determined as Live/CD3+/CD8+. Percentage of CD4 was determined as Live/CD3+/CD4+. Percentage of central memory (CM) was determined as Live/CD3+/CD45RO+/CD62L+.

Lung MILs were examined for evidence of a central memory phenotype by staining for CD45RO and CD62L. In the first experiment, a range of 64.8-74.2% central memory cells was observed and in the second experiment, a range of 67.8-75.1% central memory cells.

### Tumor Specificity

Tumor specificity was assayed utilizing the tumor specificity assay. Myeloma cell lysates were used as a negative control. Bone marrow that was autologous to the MILs product was thawed and enumerated with a hemacytometer and Trypan Blue. Cells were resuspended at 1×10⁶/ml and 100 ul were plated in a 96 well flat bottom plate. Cells were incubated for a minimum of 30 minutes 37° C./5% CO₂ to adhere antigen presenting cells (APC) to the bottom of the well. Non-adherent cells were removed with a pipette and then 100 ul of media containing either media alone, 100 ug/ml of myeloma cell line lysate (negative control) or 100 u/ml of lung cell line lysates were added to the appropriate wells to pulse the APC with the lysates.

During this time MILs autologously matched to the APC were thawed and enumerated for number and viability utilizing a hemactytometer and trypan blue. Cells were then labeled for 15 min with a 10× dilution of CFSE for 15 min at 37° C./5% CO₂ and then washed immediately with ice cold media to stop the CFSE labelling (per manufacturer's instructions). Cells are washed 2× times with ice cold media and then enumerated for number and viability with utilizing a hemocytometer and trypan blue. Cells are resuspended at 1×10⁶/ml in serum free media. 100 μl of this suspension is added to each well of the plate containing media alone, control lysate and experimental lysate groups.

Cells were cultured for 5 days. Golgi plug was added on 5 days a minimum of 4 hours before harvest. Cells were harvested from the wells and stained extracellularly for CD3, CD4, and CD8 for 10 minutes at room temperature. After extra-cellular staining, cells were permeabilized and stained intracellularly for IFNγ and sometimes also TNFα, Granzyme B and IL2, depending on the assay for 15 minutes at room temperature. Cells are washed 2× with perm wash and then resuspended in 100 μl of perm wash. The cells were run on a Beckman Coulter Navios flow cytometer and analyzed with Kaluza software. Tumor-specific T cells were defined as the IFNγ-producing CFSE-low, CD3+ population, with lung lysate percentages ranging from 2.59 in patient 1, 5.68 in patient 4, 8.77 in patient 3, and 10.05 in patient 4 (see the figure).

MILs were successfully expanded from every patient (n=4) with an average fold expansion of 123 (range: 33-194). After activation and expansion, MILs were on average 98.8% CD3+ with an approximate 2:1 ratio of CD4+:CD8+ T cells [71% to 28%, respectively].

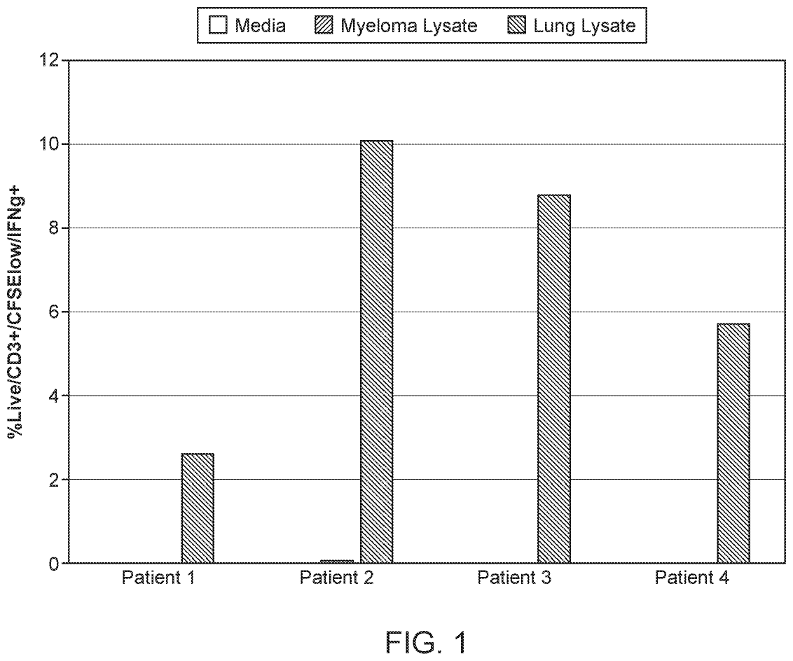

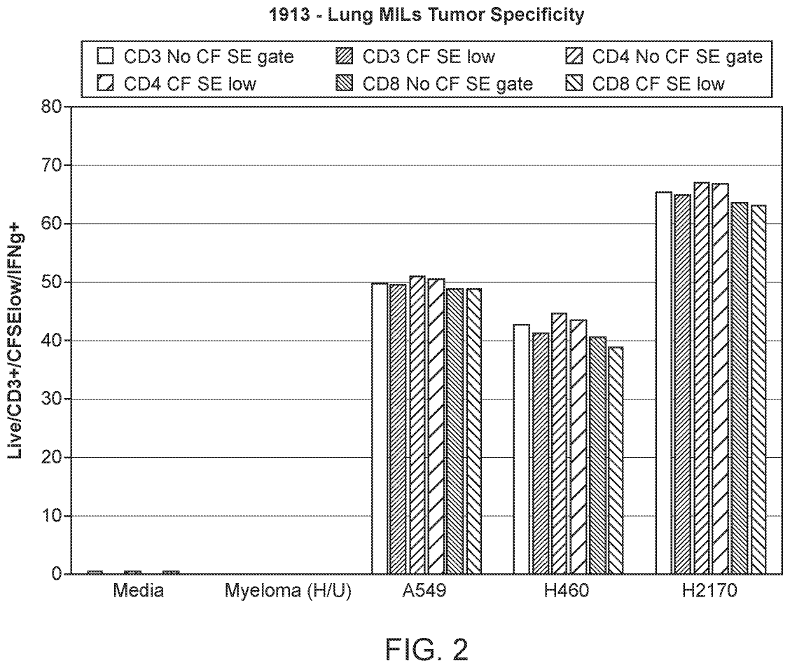

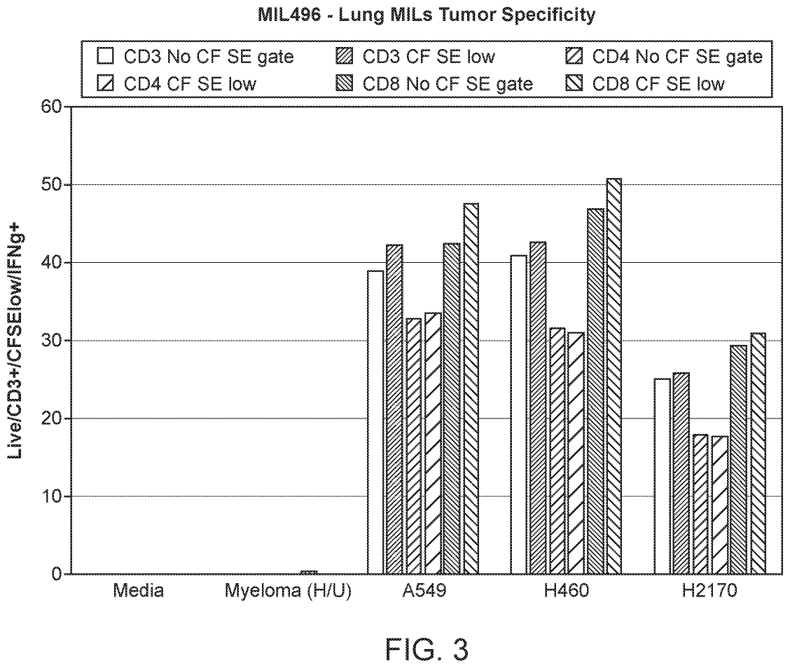

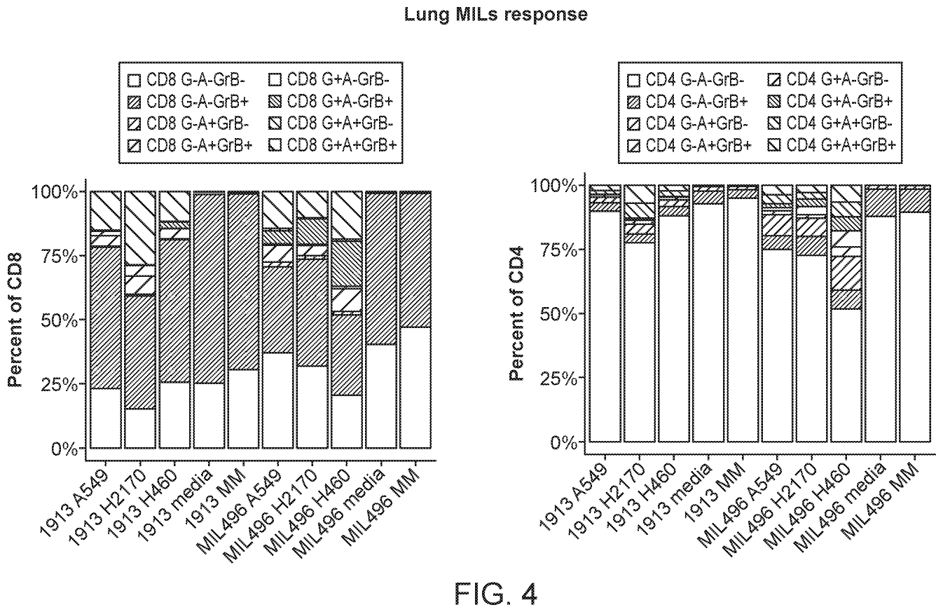

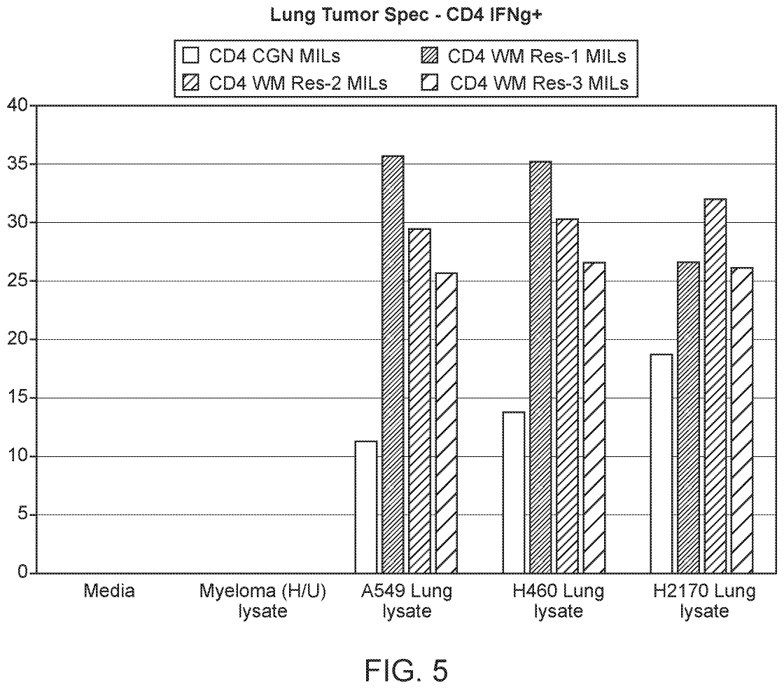

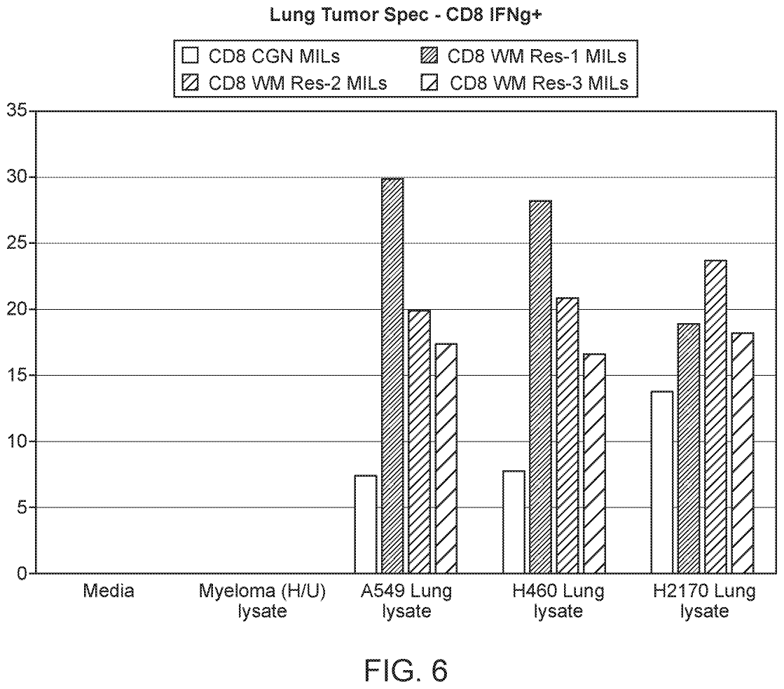

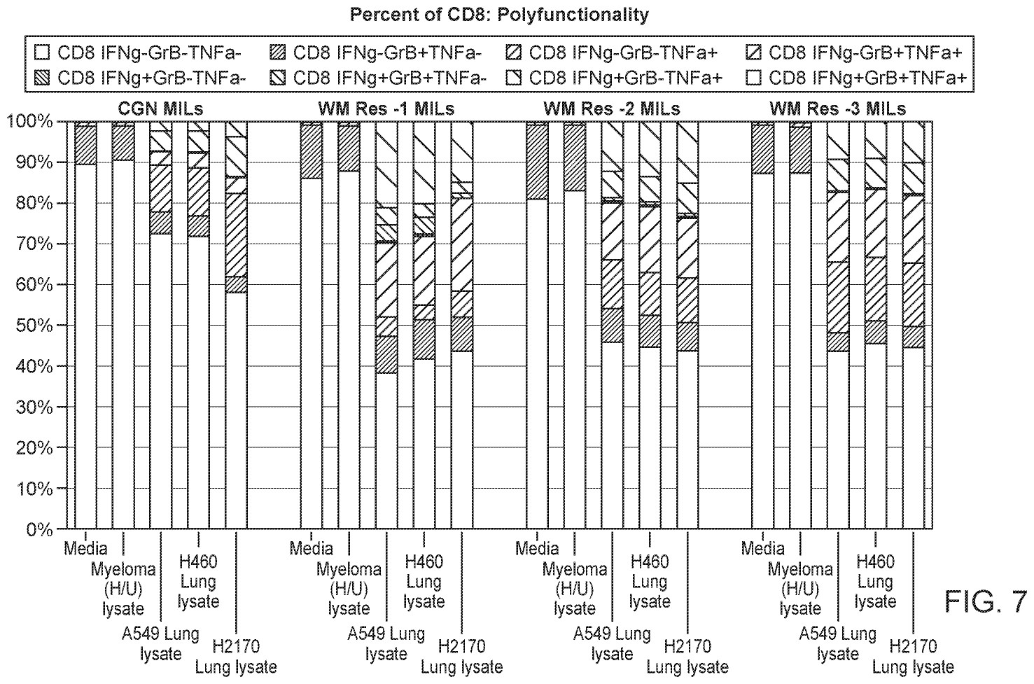

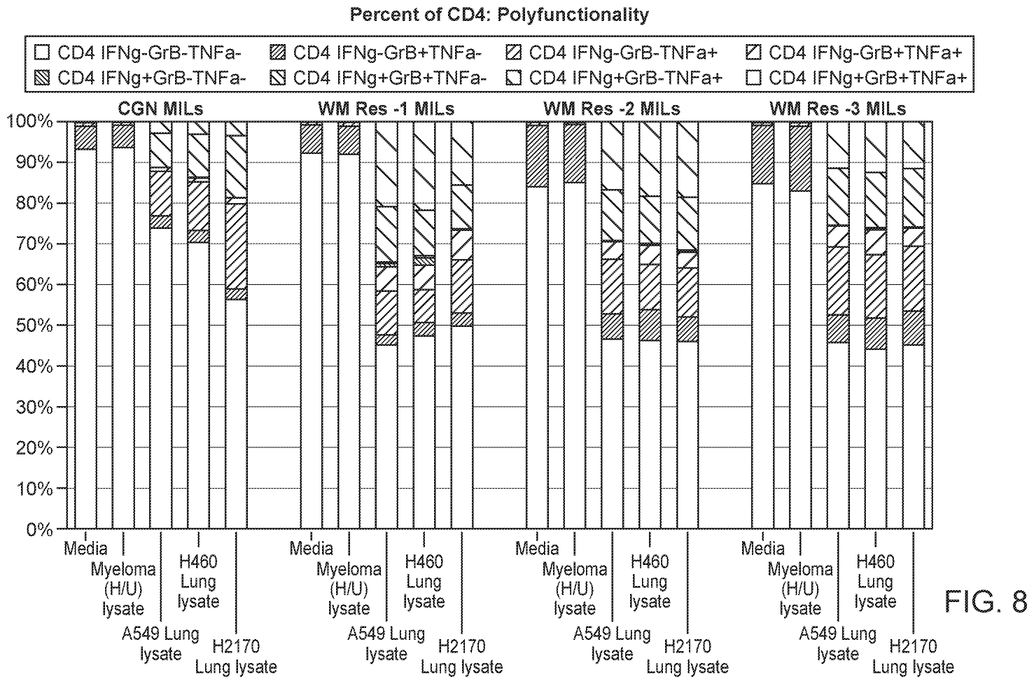

Tumor-specific T cells were detected in all of the expanded MILs products (n=4). On average, 6.8 of the total T cell repertoire were tumor specific for lung cancer antigens in the MILs products. In contrast, matched PBLs expanded and activated from four patients demonstrated no measurable tumor-specific T cells (see FIG. 1).

MILs were present and were expanded from all lung cancer bone marrow samples tested. MILs from all patients contained functionally active tumor-specific T cells. In contrast, the corresponding PBLs failed to show any detectable tumor-specific immune recognition. As such, adoptive T cell therapy with MILs as described herein is a surprising and viable novel therapeutic approach for patients with lung cancer, which until the embodiments provided for herein was not expected to be achievable using adoptive T cell therapy. Considering the central role of PD-1 to the induction of anergy, blocking PD-1 in activated T cells prior to the induction of energy is effective in prolonging and enhancing the anti-tumor efficacy of MILs. The results from the lung-specific MILs were surprising and unexpected.

### Example 2: Administration of MILs to Lung Cancer Patients

Prior to administration of MILs, patients will receive non-myeloablative lymphodepletion with cyclophosphamide (300 mg/m²/day) and fludarabine (30 mg/m²/day) from Days −5 to −3. Lymphodepletion has been shown to increase the overall efficacy of adoptive T cell therapy. 2-mercaptoethane sulfonate sodium (MESNA) may be used to minimize any bleeding in the bladder as required.

Patients may also receive pembrolizumab (200 mg) administered on Day 1 (approximately 24 hours after MILs™ administration) and again every 3 weeks.

Patients may be administered MILs™ alone. These subjects will be followed closely for 7 days post MILs™ administration for safety observation.

The MILs™ will be administered via a central catheter, which could either be a peripherally inserted central catheter (PICC) line or central line. Prior to administering the activated MILs™, the subject will be hydrated with 5% dextrose in water and 50% normal saline (D5W½NS) at a rate of approximately 200 mL per hour for at least one hour. MILs™ will be thawed at the bedside in a 37° C. (±2° C.) water bath for approximately 90 seconds (±30 seconds) per bag prior to being administered on Day 0 (+1 day). Each bag will be removed from the vapor phase liquid nitrogen shipper, one at a time, placed in the waterbath and massaged until there are some small chunks of ice present. Each bag of MILs™ will be infused at a rate of approximately 10 mL per minute and rinsed with saline prior to administering the next bag of MILs™.

Following MILs™ infusion, the patients will be hydrated with D5W½NS at a rate of approximately 200 mL per hour for 2 hours. Administration information including, but not limited to, date and time of thawing, time of administration, and infusion time, will be recorded for each bag. Dose modification is not applicable as the entire MILs™ product will be administered on at least one day, Day 0 (+1 day).

Patients will be evaluated for both measurable and non-measurable lesions via CT or MRI scans.

This description is not limited to the particular processes, compositions, or methodologies described, as these may vary. The terminology used in the description is for the purpose of describing the particular versions or embodiments only, and it is not intended to limit the scope of the embodiments described herein. Unless defined otherwise, all technical and scientific terms used herein have the same meanings as commonly understood by one of ordinary skill in the art. In some cases, terms with commonly understood meanings are defined herein for clarity and/or for ready reference, and the inclusion of such definitions herein should not necessarily be construed to represent a substantial difference over what is generally understood in the art. However, in case of conflict, the patent specification, including definitions, will prevail.

From the foregoing, it will be appreciated that various embodiments of the present disclosure have been described herein for purposes of illustration, and that various modification can be made without departing from the scope and spirit of the present disclosure. Accordingly, the various embodiments disclosed herein are not intended to be limiting. All references cited herein are hereby incorporated by reference.

1. A method for treating a subject having lung cancer with marrow infiltrating lymphocytes, the method comprising the steps of:
(a) culturing a bone marrow sample obtained from the subject having lung cancer with an anti-CD3 antibody and an anti-CD28 antibody in a hypoxic environment to produce hypoxic-activated marrow infiltrating lymphocytes;
(b) culturing the hypoxic-activated marrow infiltrating lymphocytes in a normoxic environment to produce the therapeutic activated marrow infiltrating lymphocytes; and
(c) administering the therapeutic activated marrow infiltrating lymphocytes to the subject having lung cancer.

2. The method of claim 1, wherein the hypoxic environment has an oxygen content of about 0% to about 5% oxygen.

3. The method of claim 1, wherein the lymphocytes are cultured in the presence of IL-2.

4. The method of claim 1, wherein the culturing the hypoxic-activated marrow infiltrating lymphocytes in a normoxic environment is performed in the presence of IL-2.

5. The method of claim 1, wherein the bone marrow sample is cultured in the hypoxic environment for about 24 hours.

6. The method of claim 1, wherein the bone marrow sample is cultured in the hypoxic environment for about 2 days.

7. The method of claim 1, wherein the bone marrow sample is cultured in the hypoxic environment for about 3 days.

8. The method of claim 1, wherein the bone marrow sample is cultured in the hypoxic environment for about 2 to about 5 days.

9. The method of claim 1, wherein the hypoxic environment is about 1% to about 2% oxygen.

10. The method of claim 1, wherein the hypoxic-activated marrow infiltrating lymphocytes are cultured in the normoxic environment for about 2 to about 14 days.

11. The method of claim 1, wherein the hypoxic-activated marrow infiltrating lymphocytes are cultured in the normoxic environment for about 6 days.

12. The method of claim 1, wherein the hypoxic-activated marrow infiltrating lymphocytes are cultured in the normoxic environment for about 9 days.

13. The method of claim 1, further comprising the step of removing said bone marrow sample from said subject having lung cancer prior to step (a).

14. The method of claim 1, wherein the anti-CD3 antibody and the anti-CD28 antibody are bound on a bead.

15. The method of claim 1, wherein the lung cancer is non small-cell lung cancer.

16. A method for treating a subject having lung cancer with therapeutic activated marrow infiltrating lymphocytes, the method comprising the steps of:
(a) culturing a bone marrow sample obtained from the subject having lung cancer with anti-CD3/anti-CD28 beads in a hypoxic environment of about 1% to about 2% oxygen for about 2 to about 5 days to produce hypoxic-activated marrow infiltrating lymphocytes;
(b) culturing the hypoxic-activated marrow infiltrating lymphocytes in a normoxic environment of about 21% oxygen for about 2 to about 12 days in the presence of IL-2 to produce the therapeutic activated marrow infiltrating lymphocytes; and
(c) administering the therapeutic activated marrow infiltrating lymphocytes to the subject having lung cancer.

17. A method of treating lung cancer in a subject, the method comprising administering a pharmaceutical composition comprising lung cancer specific marrow infiltrating lymphocyte to the subject.

18. The method of claim 17, wherein the lung cancer specific marrow infiltrating lymphocyte is obtained from a subject having lung cancer.

19. The method of claim 17, wherein the lung cancer specific marrow infiltrating lymphocyte is autologous to the subject being treated.

20. The method of claim 17, wherein the lung cancer specific marrow infiltrating lymphocyte is allogeneic to the subject being treated.

21. The method of claim 17, wherein the marrow infiltrating lymphocyte is hypoxic activated.

22. The method of claim 17, wherein the marrow infiltrating lymphocyte is hypoxic activated and normoxic activated.

23. The method of claim 17, wherein the pharmaceutical composition is administered by parenteral administration, intraperitoneal administration, or intramuscular administration.

24. The method of claim 17, wherein the pharmaceutical composition is administered directly into the lung of the subject.

25. The method of claim 1, wherein about 75% to about 100% of the marrow infiltrating lymphocytes administered to the subject express CD3.

26. The method of claim 1, wherein about 80% to about 100% of the marrow infiltrating lymphocytes administered to the subject express CD3.

27. The method of claim 1, wherein about 85% to about 100% of the marrow infiltrating lymphocytes administered to the subject express CD3.

28. The method of claim 1, wherein about 90% to about 100% of the marrow infiltrating lymphocytes administered to the subject express CD3.

29. The method of claim 1, wherein the ratio of CD4⁺:CD8⁺ T cells present in the composition or MILs administered to the subject is about 2:1.

30. A composition comprising a population of hypoxic-activated marrow infiltrating lymphocytes isolated from a patient with lung cancer, wherein about 75% to about 100% of the population of the hypoxic activated marrow infiltrating lymphocytes expresses CD3.

31. The composition of claim 30, wherein about 80% to about 100% of the population of the hypoxic activated marrow infiltrating lymphocytes expresses CD3.

32. The composition of claim 30, wherein about 85% to about 100% of the population of the hypoxic activated marrow infiltrating lymphocytes expresses CD3.

33. The composition of claim 30, wherein about 90% to about 100% of the population of the hypoxic activated marrow infiltrating lymphocytes expresses CD3.

34. The composition of claim 30, wherein the ratio of CD4⁺:CD8⁺ T cells present in the composition is about 2:1.

35. The composition of claim 30, wherein the cell population is obtained from a bone marrow sample obtained from a subjecting having lung cancer by:
(a) culturing the bone marrow sample with an anti-CD3 antibody and an anti-CD28 antibody in a hypoxic environment of about 1% to about 3% oxygen to produce activated marrow infiltrating lymphocytes; and
(b) culturing the activated marrow infiltrating lymphocytes in a normoxic environment in the presence of IL-2 to produce the composition.

36. The composition of claim 30, wherein the marrow infiltrating lymphocytes are lung cancer specific.

37. The method of claim 1, wherein the subject had been subjected to treatment with anti-PD-1 prior obtaining the bone marrow sample.
